(19) United States
(12) Patent Application Publication
(10) Pub. No.: US 2026/0354735 A1
(43) Pub. Date: Nov. 12, 2026

(54) ANALYZING METHOD FOR AND APPARATUS OF INTRACRANIAL DYNAMICS

(71) Applicant: OULUN YLIOPISTO, Oulu (FI)

(72) Inventors: Teemu MYLLYLÄ, Oulu (FI); Hany FERDINANDO, Oulu (FI); Vesa KORHONEN, Oulu (FI); Vesa KIVINIEMI, Oulu (FI)

(21) Appl. No.: 19/113,632

(22) PCT Filed: Jun. 15, 2023
(86) PCT No.: PCT/EP2023/066161

§ 371 Date: Mar. 20, 2025

(30) Foreign Application Priority Data
Sep. 22, 2022 (FI) 20225822

(51) Int. Cl. A61B 5/374; A61B 5/00

(52) U.S. Cl. CPC A61B 5/374; A61B 5/0075; A61B 5/4088; A61B 5/4806; A61B 5/6814; A61B 5/7257

(57) ABSTRACT

A method for measuring intracranial dynamics, by performing an optoelectronic measurement of the intracranial neuronal and fluids dynamics, brain tissue pulsation and glymphatic activity through the cranium by an optoelectronic measurement arrangement. Applying, by the data processing unit, at least one of the following analyses to electrical signals received from the optoelectronic measurement arrangement and carrying information on said dynamics. At least one of the pulses of the of the dynamics is decomposed for processing with a relation between the characteristic pulses. A moment of statistical analysis of the pulses of said dynamics is determined. A brain fluid, particularly water-hemodynamic, coupling is determined from the pulses of said dynamics based on a definition that a sum of volumes of the brain tissue, cerebrospinal fluid and intracranial blood is constant. A ratio between a power spectral density of a fraction of a whole measured frequency band and a power spectral density of the whole measured frequency band is determined. Entropy relating to the electrical signals is determined. A G-index based on said analysis of the electrical signals and a reference, which is based on an analysis of a control group with known and/or estimated intracranial dynamics, is determined, the G-index representing a relative dynamics of the brain.

### FIELD

The invention relates to an analyzing method for and apparatus of intracranial dynamics.

### BACKGROUND

The glymphatic brain clearance system, is responsible for convecting both central nervous system metabolites and waste in both brain and spinal cord. The glymphatic system maintains the brain homeostasis especially during night sleep as it balances metabolite concentrations and removes wastes that accumulate over the daily functions. Its proper activity and deviation from a normal function are known to relate to both brain health and disease conditions, respectively. Larger changes in the glymphatic system can lead to major cognitive changes.

Reduced glymphatic activity have been associated with neurodegeneration, epilepsy, narcolepsy, invasive brain tumors, ischemic strokes and several other major brain diseases. Furthermore, such as, mood, stress and sleep problems may affect function of the glymphatic system. It means the glymphatic system and brain health are related.

However, no wearable methods exist to monitor and analyze the glymphatic activity. Hence, the measurements and analyses call for improvement

### BRIEF DESCRIPTION

The present invention seeks to provide an improvement in the measurements.

The invention is defined by the independent claims. Embodiments are defined in the dependent claims.

If one or more of the embodiments is considered not to fall under the scope of the independent claims, such an embodiment is or such embodiments are still useful for understanding features of the invention.

### LIST OF DRAWINGS

Example embodiments of the present invention are described below, by way of example only, with reference to the accompanying drawings, in which

FIG. 1 illustrates an example of apparatus for measuring intracranial dynamics relating to a neuronal and hydrodynamic state;

FIG. 2 illustrates an example of an optic measurement arrangement;

FIG. 3 illustrates an example of possible approximated lines for the G-index as a function of time (age);

FIG. 4 illustrates an example of the G-index as a function of age and/or neurodegeneration based on an analysis derived from the approximated lines of FIG. 3;

FIG. 5 illustrates an example of possible approximated lines of the G-index as a function of time (day);

FIGS. 6 and 7 illustrate examples of Gaussian decomposition of brain pulsation of different brains;

FIG. 8 illustrates an example of pulse decomposition analysis;

FIG. 9 illustrates an example of a flow chart of analysis based on a distance between pulses;

FIGS. 10A to 10D illustrate examples of variation around a mean;

FIGS. 11A and 11B illustrate examples of water-hemodynamic coupling;

FIG. 12 illustrate an example of a flow chart of an analysis of the water-hemodynamic coupling;

FIG. 13 illustrate an example of a flow chart of a fractional amplitude of physiological fluctuation analysis;

FIG. 14 illustrates an example of a flow chart of a sample entropy analysis;

FIG. 15 illustrates an example of a flow chart of a spectral entropy analysis;

FIG. 16 illustrates an example of a flow chart of a cardiorespiratory envelope modulation analysis;

FIG. 17 illustrates an example of a flow chart of a phase transfer entropy analysis;

FIG. 18A to 18E illustrate examples of signal processing for spike elimination and smoothing;

FIGS. 19A to 19 C illustrate examples of a baseline correction and signal normalization;

FIG. 20 illustrates an example of a flow chart of separating individual pulses from a brain signal;

FIG. 21 illustrates an example of data processing unit; and

FIG. 22 illustrates of an example of a flow chart of an analysing method.

### DESCRIPTION OF EMBODIMENTS

The following embodiments are only examples. Although the specification may refer to “an” embodiment in several locations, this does not necessarily mean that each such reference is to the same embodiment(s), or that the feature only applies to a single embodiment.

The articles “a” and “an” give a general sense of entities, structures, components, compositions, operations, functions, connections or the like in this document. Note also that singular terms may include pluralities.

Single features of different embodiments may also be combined to provide other embodiments. Furthermore, words “comprising” and “including” should be understood as not limiting the described embodiments to consist of only those features that have been mentioned and such embodiments may also contain features/structures that have not been specifically mentioned. All combinations of the embodiments are considered possible if their combination does not lead to structural or logical contradiction.

The term “about” means that quantities or any numeric values are not exact and typically need not be exact. The reason may be tolerance, resolution, measurement error, rounding off or the like, or a fact that the feature of the solution in this document only requires that the quantity or numeric value is approximately that large. A certain tolerance is always included in real life quantities and numeric values.

It should be noted that while Figures illustrate various embodiments, they are simplified diagrams that only show some structures and/or functional entities. The connections shown in the Figures may refer to logical or physical connections. It is apparent to a person skilled in the art that the described apparatus may also comprise other functions and structures than those described in Figures and text. It should be appreciated that details of some functions, structures, and the signalling used for measurement and/or controlling are irrelevant to the actual invention. Therefore, they need not be discussed in more detail here.

The term “comprise” (and grammatical variations thereof) and the term “include” should be read as “comprise without limitation” and “include without limitation”, respectively.

In this application, the term “determine” in its various grammatical forms may mean calculating, computing, data processing for deriving a result, looking up in a database or the like. As a result “determine” may also mean select, choose or the like.

The abbreviation HbO refers to oxygen haemoglobin, the haemoglobin being measured in blood. The abbreviation HbR refers to haemoglobin with reduced oxygen, the haemoglobin being measured in blood. The abbreviation HbT refers to a total oxygen of the blood or total haemoglobin A person skilled in the art is familiar with these terms, per se.

The term “very low frequency (VLF) band” refers to a frequency band about 0.008 Hz to about 0.1 Hz.

The term “respiratory band” refers to a frequency band for recoding breathing. The respiratory band is about 0.1 Hz to about 0.6 Hz.

The term “cardiac band” refers to a frequency band for recording heart rate. The cardiac band is about 0.6 Hz to about 5 Hz with harmonics of the principal cardiac frequency. The given frequency ranges refer to typical physiological frequency ranges in human.

The glymphatic system including brain fluids are responsible for brain cleaning from metabolic waste and it should work properly to maintain the brain health. Its activity and condition are related to the brain health and brain condition and thus also to neurodegenerative diseases (NDDs). The NDDs are mainly found in the elderly which implies that the brain function is affected by NDD and vice versa. Larger changes in the glymphatic activity of the brain are responsible for cognitive changes. It means the glymphatic activity of the brain of particularly the elderly with and without NDD should differ from each other. Normal aging without any evidence of mild cognitive impairment (MCI) or dementia changes cognitive capacities as well and it typically slowly starts after age of about 40 as the brain starts to lose volume, weight and tissue elasticity. Prefrontal cortex is one of the brain areas affected by aging and is responsible for memory storage. Two main other areas are posterior associational neocortex and medial temporal lobe. It means two different age groups should have different glymphatic activities of the brain which may also appear as different brain health.

Sleep disorders causing mood disorders and stress are also associated with the glymphatic system. It means the glymphatic activity reflects, in addition to brain health, also mental health.

The glymphatic system activity reflecting the clearance mechanism, which can be monitored using the apparatus described in this document, uses neuro-hydrodynamical or neurofluid (i.e. blood, cerebrospinal and brain tissue intestitital fluids) and mechanical pulsations of the brain to assess the glymphatic activity and brain clearance. The glymphatic system clears metabolic waste and balances reduced metabolites of the brain especially during sleep, and an improper glymphatic convection may decrease wellbeing of mammalians such as human beings. Additionally, a failure of the glymphatic convection has even been connected to several major brain disorders including Alzheimer's disease, fronto-temporal dementia, vascular dementia, normal pressure hydrocephalus, multiple sclerosis, epilepsy, trauma, stroke, tumors, hydrocephalus, Chiari malformation, syringomyelia, pseudotumor cerebri, cerebral vasospasm, glaucoma, cerebral aneurysms or the like for example. Although the dynamics of the brain and neurofluids within the cranium can be measured and monitored using the apparatus described in this document, determination and/or diagnosis of any potential disease based on the measurement results expressly remains to a medical personnel. Namely, the measured dynamics may be based on the heartbeat and/or the respiration and/or low frequency vasomotor waves (Mayer & Traube-Hering waves).

An optoelectronic measurement system of this document can measure and monitor volume dynamics of the brain and brain fluids within the cranium. The fluids within the cranium include interstitial fluid, CSF (CerebroSpinal Fluid) and blood. The measurement of the permeable movement of these fluids between blood, perivascular glymphatic CSF and brain tissue interstitial fluid may be performed with the full band direct-current (DC) electroencephalography (EEG) also involving electrophysiological activity from about 0 Hz to about 1000 Hz” The optoelectronic measurement system and EEG may jointly be used to quantify hydrodynamics The study of glymphatic system and neurofluid dynamics is a fairly new branch within neurophysiology and the recorded when processed as described in this document is used to form a parameter or index value that describes the function of the brain that can be called in more detail glymphatic activity, brain clearance activity, brain fluid dynamics, neurofluid dynamics or neurohydro dynamics of the brain. All in all, it can be considered to be a question of intracranial dynamics including neuronal and fluid dynamics, brain tissue pulsation and glymphatic activity related to the neurological central system.

By using a light source with at least three wavelengths in near-infrared range (NIR) range (one below 800 nm, one between 800 nm and 940 nm and one above 940 nm, for example), and at least one photo detector for these wavelengths, brain tissue dynamics and/or fluid dynamics can be measured. Additionally, blood/haemoglobin dynamics may be measured. The brain tissue water/or neurofluid dynamics can be seen as pulsatile volume/or concentration/or flow changes, which is caused mainly by three leading physiological pulsation sources: cardiorespiratory and vasomotor pulsations. Each of the pulsation source has a different, non-overlapping frequency range that can be used to identify the pulsation source. This further can be used to identify the measured brain fluid compartments and importantly to identify failures of the glymphatic clearance mechanisms. In addition, movements or vibration of head can be used as a pulsation source and the movements can be measured using an acceleration sensor, for example. Different near-infrared (NIR) light wavelengths result in specific intensity changes or pulse shapes of the detected light, and this shape can be used to both identify neurofluid compartment and to estimate continuously changes related to brain stiffness or elasticity and intracranial pressure that may all affect the glymphatic activity.

Using additionally a direct current EEG (DC-EEG, ElectroEncephaloGram) arrangement with at least two electrodes, it is possible to measure electric potential and time domain signals between brain tissue interstitium and blood for direct information on glymphatic fluid (or water)/electrolyte permeable movement over in the glia limitans interface between blood and brain tissue, i.e. from the glymphatic paravascular space. Interaction between the gathered signals, in particular their specific phase differences, and amplitude changes reflect activation of the glymphatic system and blood-brain barrier (BBB) permeability, and CSF flow dynamics between CSF and brain tissue. Moreover the direction of information flow between the signals can be used to quantify awareness connection to neurofluid changes.

An apparatus of this document can be used to measure and analyze the signals from the brain and their interactions. The apparatus of this document may be wearable, and it can be used for long-term monitoring of the glymphatic activity and examining the well-being of the brain.

Glymphatic brain fluid convection and clearance precedes several chronic brain diseases such as Alzheimer's disease, chronic traumatic encephalopathy, and in tumors and focal epilepsies scar formation prevents the normal CSF convection. After bleeding or arterial ischemic occlusion CSF (or brain water) may flow into the brain tissue inducing edema and increased pressure. The glymphatic system is similar in mammals including humans and mice as it has been shown to be similar with magnetic resonance imaging with invasive gadolinium agent (MRI Gd3+) intrathecal injections and contrast media imaging lasting 24-48 h.

Currently there are no systems that determine the glymphatic activity of the brain of a subject such that the result could be compared between people of groups.

What is presented in this document enables easy monitoring of sleep quality and can quantify the glymphatic increase related brain clearance increase in sleep and importantly monitor the subject also in upright position in daily activity. Further, it provides a method to study and diagnose how, such as, physiological exercise and different treatments affect glymphatic system and brain clearance and wellbeing of the mammal such as a human being.

The monitoring may be performed during awake in a short measurement time up to 10 minutes, or during overnight sleep, for example, which can easily be done with the wearable support structure 102 having at least sensing device 100. Additionally, the measurement results may be used in conjunction of diagnoses of brain diseases such as Alzheimer's disease, stroke, Parkinson, epilepsy, tumors, etc. but that is the work of the medical personnel.

FIG. 1 illustrates an example of a measurement apparatus for measuring the intracranial dynamics i.e dynamics of the neurological central nervous system and/or intracranial neuronal and fluid dynamics, and glymphatic activity, the apparatus comprising at least one sensing device 100. The apparatus for measuring the intracranial dynamics may comprise a support structure 102 to which the least one sensing device 100 is attached. The support structure 102 may be a band round the head or a cap on the head, for example, without limiting to these. Because the support structure 102 with the at least one sensing device 100 is easy to wear on the head, the apparatus is literally wearable. The at least one sensing device 100 transmits in a wired or wireless manner information of the intracranial dynamics to a data processing unit 150 which may be located at a distance from a mammal 10 such as a human being that has the at least one sensing device 100 attached to the head.

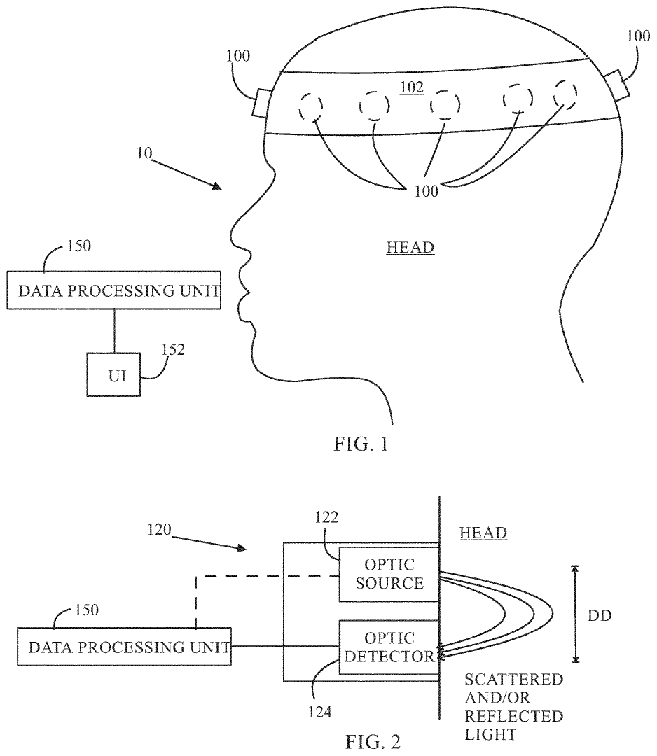

At least one sensing device 100 is an optic measurement arrangement 120, an example of which is illustrated in FIG. 2. The optic measurement arrangement 120 may comprise at least one optic radiation source 122 and at least one optic radiation detector 124. Each of the at least one optic radiation source 122 directs optic radiation toward the brain through the cranium, and the at least one optic detector 124 receives the optic radiation reflected and/or scattered therefrom. The at least one optic radiation source 122 may be in contact with the skin of the cranium. The at least one optic detector 124 may be in contact with the skin of the cranium. Alternatively, there may be a non-zero distance between the at least one optic radiation source 122 and the skin of the cranium. Correspondingly, there may be a non-zero distance between the at least one optic detector 122 and the skin of the cranium.

When light penetrates into tissue, it starts to scatter and attenuate. This event is also affected by partly irregular physiological events, such as breathing, muscle movements and blood flow. The result is that intensity changes in the frequency spectrum of the received light exhibit variation which may be constant and/or nonlinear. Most importantly, these intensity changes also include wavelength-dependent variation that can be exploited to monitor metabolism and brain activity. Oxy-haemoglobin and deoxy-hemologlobin may be detected.

An fNIRS (functional Near InfraRed Spectroscopy) signal cardiac activity related pulsation recorded from the brain is similar to the PPG (PhotoPlethysmoGram) signal recorded from the skin or finger, with a steep upslope and slow decaying downslope. Each pulse represents a single heartbeat, and its shape varies from beat to beat. This can be called brain pulsation, since whole brain is pulsating due to cardiac activity when blood volume is constantly changing within the blood vessels which results in pulse shape changes due to blood flow and blood pressure propagation through the blood vessels. Consequently, and importantly, because the pulse shape is also dependent on the tissue properties. As the brain shrinks due to volume and weight lost, the properties of brain tissue also changes and affects the brain tissue and vessel elasticity, and its pulsation.

The data processing unit 150 can apply one or more analyses to electrical signals that it receives from the optic measurement apparatus 120, the electrical signals carrying information on the dynamics of the neurological central nervous system in forms of pulses of the brain pulsation.

The analysis may be based on time domain analysis and/or frequency domain analysis. The time domain-based analyses are pulse shape analysis (or decomposition analysis), water-hemodynamic coupling and water-EEG coupling (interconnections) analysis and sample entropy, for example. In time domain-based analyses distances between hemodynamic, water and EEG pulses (neural activations) may be determined at each cardiac pulse, for example. Here intracranial water (H2O), or brain water refers also to cerebrospinal fluid (CSF). In addition, differences between these couplings can be analysed between different measurement locations in the brain, in particular correlations between the hemisheres. For instance, interhemispheric balance analysis within cardiac band, 0.6-5 Hz, on HbO-H2O and HbR-H2O concentrations measured in resting state from prefontal core area delivers good separation between stroke patients from controls.

Moreover, hemo-water dynamics in very low frequency (0.008-0.1 Hz), respiratory (0.1-0.6 Hz), and cardiovascular (0.6-5 Hz) bands provide good separation between AD and controls when the hemodynamic-water couplings is cross correlated within a sliding time window. The lags form a series, which are subject to statistical measures as features to differentiate AD patients from control based on a t-test at 0.05 significant difference. In particular, SD and IQR of the series in cardiac band provides the best separation delivered by HbR-H2O pair, e.g. characterised by IQR with p-value between 0,0002 and 0.0018.

The frequency domain-based analyses are fractional amplitude of physiological fluctuation (fAPF), power spectral density of high and low band, spectral entropy, cardiorespiratory envelope modulation (CREM),

Additionally, the following analyses may be done: tissue oxygenation, phase locking value, phase transfer entropy and EEG signal analysis.

The phase transfer entropy forms dPTE (normalized values) and PTE values. The following pairs may be evaluated: HbO-HbR, HbO-H₂O, HbR-H₂O, HbT-H₂O, and EEG-H₂O. Moreover, simultaneous multi-directional analyses between the dcEEG, H2O, HbO, HbR can provide more accurate information on the causal relations between the water permeability of BBB and neuronal interactions.

Using dPTE (normalized values), a transfer from both directions gives the same p-value.

Using PTE, transfer from both directions may have different p-value, it even changes from significant to non-significant and vice versa

Significant results from 10 Hz:

HbO-HbR at cardiac band (L) A 0.0277

HbT-H2O at respiratory band (L) à 0.0215

Significant results from 800 Hz:

HbO-HbR at VLF band (L) à 0.0279

HbO-H2O at VLF band (R) à 0.0420

HbO-H2O at respiratory band (L) à 0.0486

Mathematically this can be expressed as:

dPTE_{x1→x2}=nPTE_{x1→x2}−nPTE_{x2→x1}.

Here the non-integer numbers after the symbol “à” refer to a p-value which indicates a probability of error. When the p-value is below 0.05, the result is significant.

It should also be noted that fNIRS and EEG signal analysis may be done together and a combined result may be formed.

In addition, we calculate power spectral density (PSD) using periodogram on the selected band, VLF (0.008-0.1 Hz), respiratory (0.1-0.6 Hz), and cardiac (0.6-5 Hz) bands. Then, the fAPF feature for each band is calculated by dividing PSD from corresponding band with that from the whole band. We calculate fAPF feature for HbO, HbR, and HbT. Similarly, spectral entropy (SE) features are also calculated from all concentration, HbO, HbR, and HbT. SE features from all concentrations in VLF band provide good separation between AD patients and controls. In addition, SE features from VLF band provide very low p-value, indicating their significant difference between patients and controls.

The fNIRS measuring cerebral pulses can be analysed by suitable statistical methods that distinguish subjects from each other when the subjects have different glymphatic activity. Additionally or alternatively, each of the fNIRS pulses can also be decomposed into several characteristic pulses, such that the sum of the characteristic pulse reconstructs the original one. Among many choices of characteristic pulses, a log-normal pulse may be selected. The log-normal pulse consists of three parameters, amplitude, position, and width. A single fNIRS pulse can be decomposed into characteristic log-normal pulses a number of which may be adaptive. By decomposing a single fNIRS pulse into five characteristic log-normal pulses, for example, fifteen parameters are collected. The pulse decomposition analysis can be used to classify subjects based on age group, obesity, and hypertension, vessel and brain stiffness, for example. The characteristic pulses may also be referred to as standard pulses.

From an fNIRS signal, concentration changes of oxy- and deoxy-haemoglobin, HbO and HbR respectively, and water may be calculated using the modified Beer-Lambert law (MBLL), which is a known method, per se. Total haemoglobin (HbT) as a sum of HbO and HbR may also be formed. They represent concentration changes under the fNIRS sensor. From either raw fNIRS signal or HbO, HbR, HbT, and water, the G-index may be formed.

Another example of the at least one sensing device 100 is an electrode of an electroencephalographic (EEG) electrode arrangement, which comprises the support 102 and at least two electrodes. The EEG measurement can be used in addition to the optical measurement. The at least one sensing device 100 is in an electric contact with skin of a cranium of the mammal 10 an example of which is a human being. The sensing devices of the electroencephalographic electrode arrangement receive and sense direct-current (DC) electroencephalographic signals from the brain of the mammal 10. A person skilled in the art is familiar with the electroencephalographic electrode arrangement, per se, although the used bandwidth of the DC EEG is unusual, being in a range about 0 Hz to about 0.5 Hz.

In an embodiment, the at least one optic radiation source 122 may be pigtailed such that the optic radiation is guided from the at least one optic source 122 to the skin of the cranium through an optic fiber. In an embodiment, the at least one optic detector 124 may be pigtailed such that the optic radiation is guided from the skin of the cranium through an optic fiber to the at least one optic detector 124. The optic fiber end(s) may be in contact with the skin of the cranium or be in a non-zero distance from the skin. The optic fibers are not separately illustrated in Figures.

In an embodiment, all the sensing devices 100 may be optic detectors. In an embodiment, the sensing devices 100 may comprise a combination of at least two of different kinds of sensing devices 100. A combination may include at least one electroencephalographic sensing device 100 and at least one optic sensing device 100.

A data processing unit 150 receives the electric signals from the at least one sensing device 100. The electric signal may be filtered, and their baseline may be corrected and/or stabilized. A person skilled in the art is familiar with the existing filtering and baseline correction/stabilization methods, per se.

The data processing arrangement 150 then processes the electric signals and forms a G-index, which represents the glymphatic activity of the brain referred to earlier in this document.

### Calculation of the G-Index

The G-index represents the glymphatic activity of the brain referred to earlier in this document. The G-index is based on one analysis or a combination of various analyses of the brain signal performed by the data processing arrangement 150. The G-index can be calculated from the fNIRS measurement alone or as a combination of the EEG measurement and the fNIRS measurement. The calculations may utilize a single- and/or multimodal approach, the multimodal approach including a plurality of analyses of the brain signals of either the fNIRS measurement or a combination of the fNIRS measurement and the EEG measurement.

One of the multimodal analyses is coupling between different G-index values of different analysis, because any two analyses relate to different physiological phenomenon in principle. Thus, a relationship among several phenomena might reflect interesting physiological conditions. This kind of multimodal approach may provide a better and wider scope than single modality which gives a G-index based on a single analysis method. However, also a single modality and a single feature of the signal, or algorithm, can be used as the G-index value.

The rate of change and the trend can vary depending on the subject(s) or condition(s); thus, more than one models may be used, the models being based on groups of known glymphatic activities. Different models also allow different analysis methods.

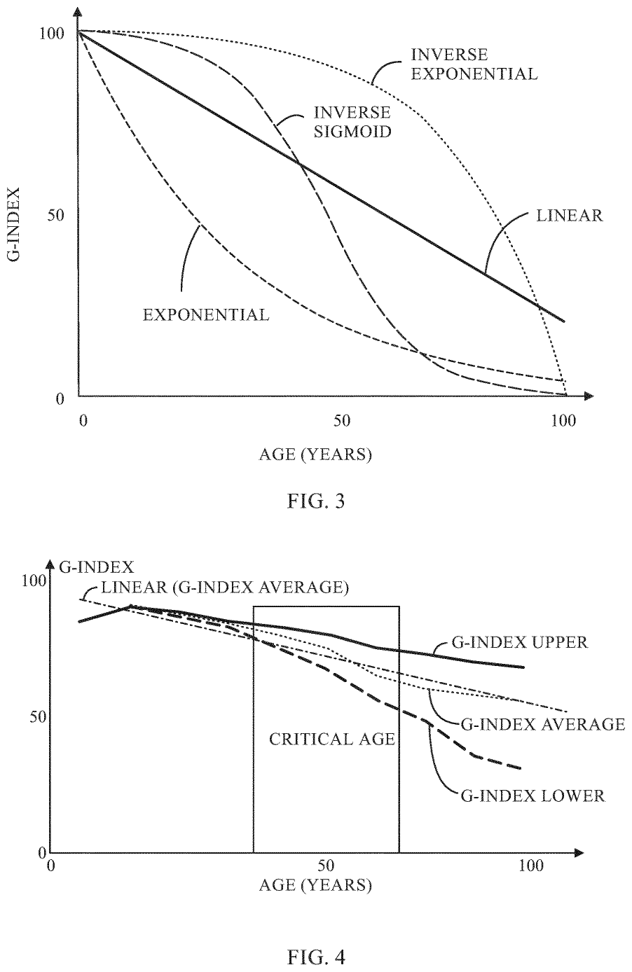

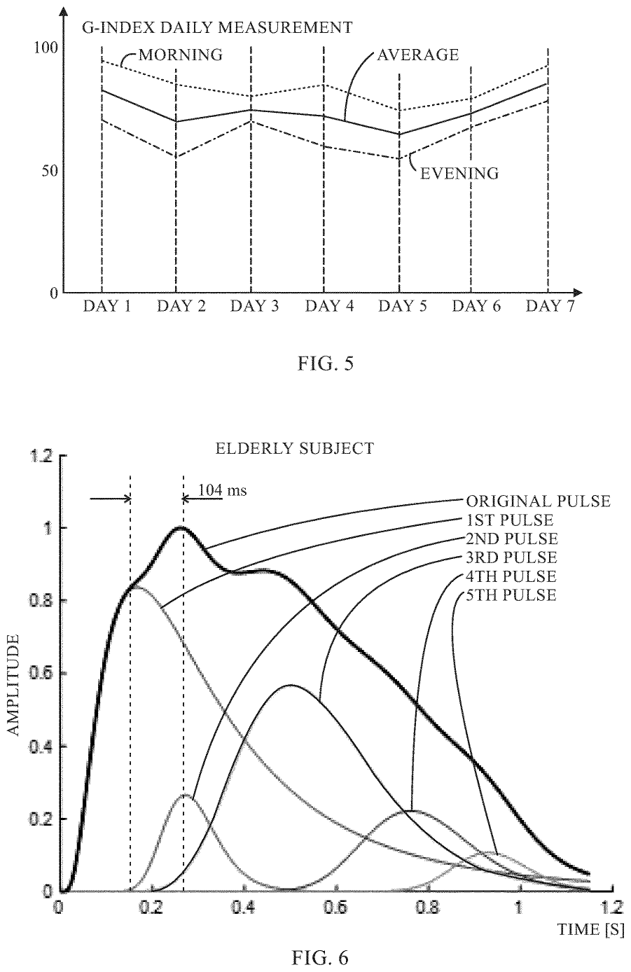

Possible approximated lines of the G-index as a function of time are shown in FIG. 3. In FIG. 3, the time axis is age in years. The rate of change and the trend can vary depending on the subject(s) or condition; thus, several models are proposed. The approximated lines can be used to predict G-index during G-index model development process. Parameter k in a linear function (−kx) or an exponent function (e^{-kx}) modelling the G-index can be adjusted for a suitable match with the measured results, for example, where x is time such as age or related to age. The matching may be performed using a smallest square error, for example. The slope is negative as a function of age, particularly for adults.

Both single- and multimodality analysis models can be utilized. The brain signals can be measured from subject of brain conditions and subjects each having a healthy brain. The experiments can utilize brain signals measured from both Alzheimer's Disease (AD) patients and a control group (healthy persons of the same age range), aiming to choose suitable indices for the G-index. The control group may be used as a reference although any group with or without brain conditions may be selected as a reference. General guidelines are required for deciding which indices are good candidates. The following guidelines may be used to evaluate whether the proposed methods are promising for a further processing:

1. They should be able to separate patients from age-matched controls

2. They should be able to separate healthy controls with same age but different living styles and e.g., when having differences in sleep quality, stress, smoking, eating/diet etc.

3. They should provide good separation among subjects with different ages.

All proposed methods should be evaluated using simple t-test at 0.05 significant level. The methods are promising when they result in said three separations.

There are several methods that fulfill the criteria. It can also be defined that there are several modalities where differences in the G-index exist and can be observed and at least one of those methods can be utilized. In an embodiment, two or more indices from different methods may be combined to estimate the G-index. Combining different indices may be done by assigning a certain weight value for the corresponding indices. For example, the G-index may equal to 100 or some other desired value may represent the best brain health or most desired glymphatic activity, while zero or some other desired value may be the worst, see FIGS. 3 and 4. The G-index is inversely proportional to the age. In addition, the G-index of healthy persons at certain age, should be higher than that of patients suffering from a condition affecting the brain. Within healthy persons, the G-index from different lifestyle may be different as well. Experiments to develop the G-index model may be based on these assumptions.

The data processing unit 150 processes the electrical signals it receives. The electrical signals carry information on the brain and particularly glymphatic activity in their pulsation. Each of the pulses can be processed using decomposition, statistical methods, and a power distribution. The pulses can alternatively or additionally be decomposed into a group of pulses and form the G-index based on one or more properties of the decomposed pulses. Additionally or alternatively, the processing in the data processing unit 150 may utilize one or more moments of the statistical analysis of the decomposed pulses or the non-decomposed pulse. The statistical analysis by be based on mean, variance, skewness, and kurtosis. The data processing unit 150 may then form the G-index based on them. The non-decomposed pulse may be so called raw pulse or it may be filtered for reducing noise, interference and/or artefacts.

The mathematics of the analysis methods is known, per se. However, they are now specially applied to the electric signals received from the optical measurement of the brain. They may also be applied to the EEG measurements.

The pulsations of the brain have been modelled for various groups of people that have different brain function i.e. different G-indices. Different groups can have different age range and within age groups people may be divided into groups that have healthy brain and one or more brain conditions such as Alzheimer and/or mental health issues, for example. The conditions, in turn, may be sub-divided based on severity of the condition and so on.

### Modelling

### Phase 1

It is necessary to conduct feature analysis to see which features have good contribution using, e.g., principal component analysis (PCA). It also serves as a dimensionality reduction to speed up calculation. Then, a simple machine learning classifier, such as kNN (k-Nearest Neighbour) or multi-class support vector machine can be used as the learning algorithm, for example.

Data collection may be performed as follows:
Data is collected from healthy subjects who are 20 years old or older.
Measurement time is 10 minutes per session for the sake of analysis in VLF band (0.008-0.1 Hz)
Subjects are measured for several sessions on different week because it can be hypothesised that G-index should be dynamic but within certain ranges
Subjects are measured on resting state in sitting position
Subjects should restrict themselves from certain medications and food that can alter the measurement

### Data Processing:

All signals may pass standard signal processing procedure, e.g., reducing artefact, selecting the band of interest, etc.
Features are extracted using the promising methods.
Make a group based on the age: 20-29, 30-39, 40-49, 50-59, older than 59, for example
Each group can now be assigned with a label for classification
Apply machine learning analysis protocol with learning algorithms, e.g., k-nearest neighbor (kNN), support vector machine (SVM), neural network (NN), for classification purpose.
A validation like k-fold cross validation or nested cross-validation may be used, but the isolated data is from the same week and from several subjects of different ages. The isolated test dataset consists of subjects whose measurement data is unseen by the trained model.

### Evaluation Criteria:

Sets of models, which result in high accuracy or similar high performance with other appropriate performance metrics such as F1-score, sensitivity, specificity or balanced accuracy, are useful for further exploration. The set of features for further analysis may be selected according to the models with high achieved performance with the developed model or by suitable feature importance analysis.

During feature extraction, many features, which are more or less useful, are obtained. Of them suitable ones are selected to support the goal properly.

### Phase 2

Based on the G-index model phase 1, a regression model can be developed in phase 2. The aim of this phase is to get real value for each subject, instead of classifying subjects to corresponding age group. The model development starts from the simplest regression, i.e., linear regression. Other types of regression, e.g., logistic regression, logarithmic regression, may also be evaluated.

### Data Collection:

There is no data collection in this phase because all required signals have been collected during the phase 1.

Data processing may go as follows, for example:
Sets of features with selected in the phase 1 are used in this phase
For each group, the following “predicted” G-index values are assigned.

An idea may be to have a high G-index for young subjects. Proper values for each group are not known yet, so the “predicted” values are used temporarily. These predicted scores are derived based on the linear approximation
20-29 years old: 70
30-39 years old: 60
40-49 years old: 50
50-59 years old: 40
Older than 59 years old: 30
Apply machine learning algorithms that result in a real value instead of class or label, e.g., regression (linear, logarithmic, logistic, etc.) may be applied, such as Bayesian network, support vector regression, etc.
A model may be validated based on the isolated data from certain week, which consists of data of several subjects of different ages. The isolated test dataset consists of subjects whose measurement data is unseen by the trained model
Apply “predicted” G-index also to the other approximation lines, see FIG. 3, and other type of regression algorithm. If necessary, the parameters in the approximation line can be changed to get reliable and credible results

### Evaluation Criteria:

The result should be similar to that of FIG. 4
Mean squared error (MSE) or other suitable performance metric may be used for validation with good performance.
The variation among different measurements on the same subject should be within about mean±2 times standard deviation, for example.

### Expected Results:

Rough idea about the approximation line for a G-index curve and the parameters.

### Phase 3

In phase 3, subjects from children and teenagers may also be involved. The process is the same as the G-index model phase 2, and may be conducted for example as follows

### Data Collection:

Subjects:
Teenager: 13-19 years old
Children: 7-12 years old
Small children: 2-6 years old
Data collection is conducted with the same protocol

### Data Processing:

Combine signals from these subjects with the previous data collection in phase 1.
Use the same procedure in phase 2.
Assign the following “predicted” G-index from linear approximation:
2-6 years old: 100
7-12 years old: 90
13-19 years old: 80
Apply “predicted” G-index using the other approximation lines, see FIG. 3.

### Evaluation Criteria:

See phase 2

### Expected Results:

Rough idea about the approximation line for G-index curve and the parameters after adding data from children and teenagers.

### Phase 4

In phase 4, patients with certain NDDs are involved and the processes follow phases 1 and 2. This phase requires more data collection from patients with standardized NDD assessment. At this point, we ignore the severity of NDD and the age. It means as long as patients have a low G-index, it is fine. The G-index from healthy subjects should have similar performance as in phase 3. The phase 4 may be conducted for example as follows.

### Data Collection:

Data is collected from patients with NDDs.
Patients go through examination to determine how severe their health condition.
Data collection is conducted with the same protocol

### Data Processing:

Data from patients is used in two scenarios: process all patients as one entity (patients with NDDs) and process data based on the NDDs.
The predicted G-index for this group is either 10 or 20 based on the linear line approximation. These choices will be investigated in this phase.
Combine data from patients with data in phases 2 and 3
Use the same procedure as in phase 2

### Evaluation Criteria:

See phase 2

### Expected Results:

The approximation line for G-index curve and the parameters after adding data from patients.

### Phase 5

In phase 5, the severity of the NDD is involved. Patients are categorized based on the degree of severity of the NDD. For this experiment, we ignore their age. This data is combined with data used in phase 4 to build the next model. The processes follow phase 4. The phase 5 may be conducted for example as follows.

### Data Collection:

There is no data collection in this phase because it is done in the previous phase

### Data Processing:

Due to variation of severity level, data from patients is divided into three groups
The “predicted” G-index values are 10, 5, 0 based on the linear approximation line.
Repeat procedure in phase 1, but only for data from the NDD, for each of the NDDs.

### Evaluation Criteria:

See phase 1. If the performance is promising, procedure in phase 2 can be executed.

### Expected Results:

The approximation line for the G-index curve and the parameters with gradual changes to indicate the severity level of NDDs.

In general, the G-index model development involves the following algorithms:
Classification:
kNN using various distance measure, e.g., Euclidean, Mahalanobis, Chebyshev, city block or the like for example
supervised learning algorithm such as the support vector machine (SVM, using various kernel, e.g., linear, polynomial, radial basis function, and sigmoid)
Tree and random forest
Regression:
Regression
Support vector regression
Bayesian network

One or more of these models are available for the data processing unit 150 as a reference for formation of the G-index as function of age or some other parameter in order to describe a person's glymphatic activity of the brain or the brain health with respect to the reference. The reference may be a G-index of a normal, healthy brain, for example. Alternatively, the reference may be a G-index of a group of people with a brain condition or a deviance from a normal brain function caused by lifestyle, medication, sickness, obesity, stress, mental health, pregnancy, injury, nutrition, or the like, for example.

### Processing Measured Signals

In an embodiment, the dynamics of the neurological central nervous system may be measured in a frequency band about 0 Hz to about 0.5 Hz. In an embodiment, the dynamics may be measured in a frequency band about 0 Hz to about 2.5 Hz. In an embodiment, the dynamics may be measured in a frequency band about 0 Hz to about 4 Hz. In an embodiment, the dynamics may be measured in a frequency band about 0 Hz to about 5 Hz. Dynamics of glymphatic system refers to the pulsatile activity related to its clearance function.

### Decomposition

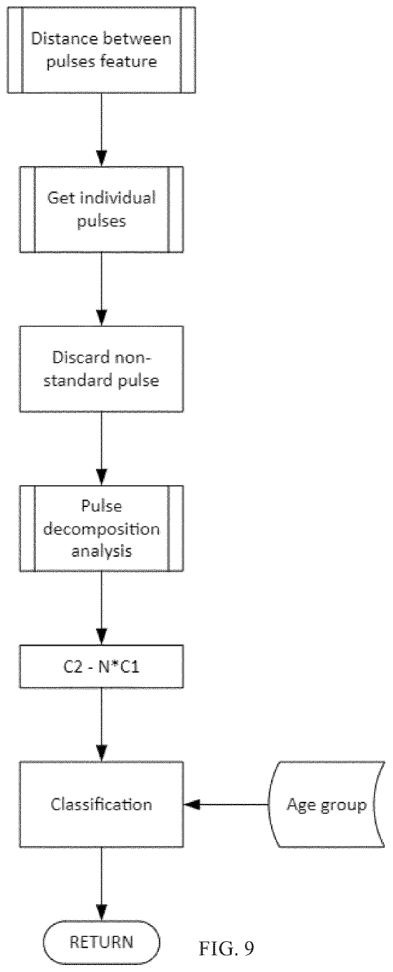

Examples of analysis based on decomposition are illustrated in FIGS. 6, 7 and 9. A waveform of the received pulse may be decomposed based on an integral transform. In an embodiment the integral transform may be the Fourier transform or the Laplace transform, which the person skilled in the art is familiar with. The data processing unit 150 can decompose at least one of the pulses of the of the dynamics of the neurological central nervous system into a plurality of characteristic pulses for processing at least one relation between the characteristic pulses such as a temporal distance between the characteristic pulses.

In general, a pulse may be converted into a pulse that looks like a Gaussian pulse. A natural logarithm without limiting to this can be used to perform a log transform. Scaling of the variable may be needed because negative values are not allowed (logarithm does not have a real value for negative numbers). Scaling if needed may be performed in conjunction with filtering, for example.

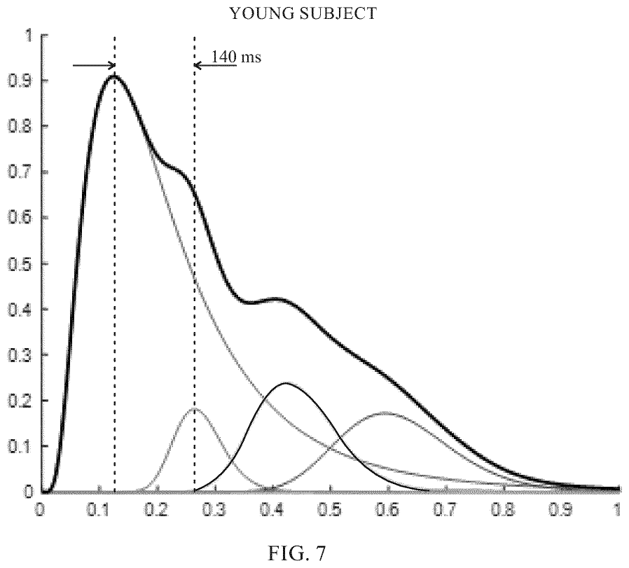

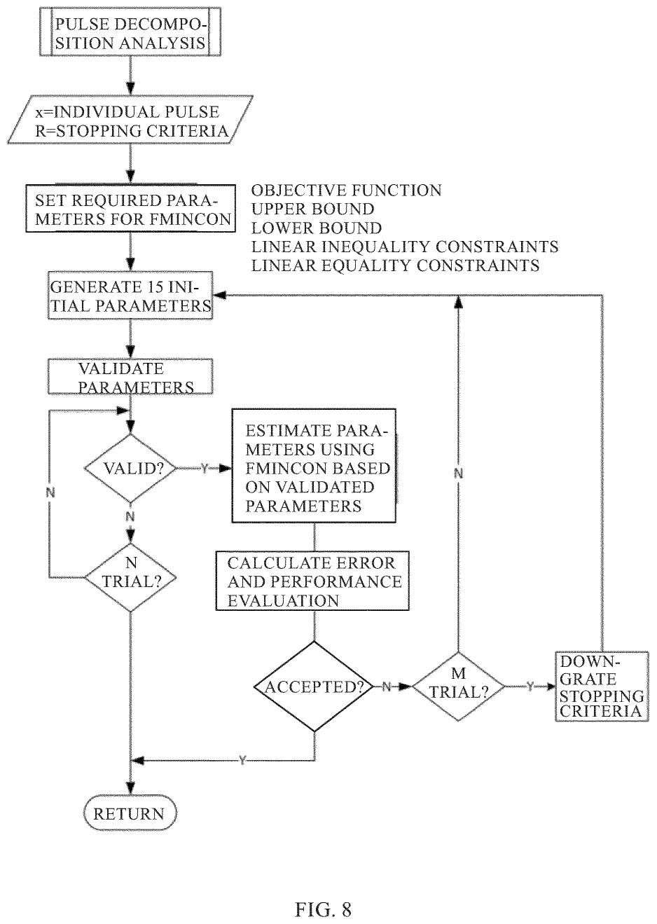

Using a transform, an example of which may be a Gaussian decomposition, 3 to 5 characteristic individual log-normal pulses or waves may be extracted, for example. The number of the characteristic pulses may be adjustable for the measurement. There are 5 characteristic log-normal pulses as shown in FIGS. 6 and 7. The Gaussian decomposition may be expressed mathematically as

$$f ( A , c , σ ) = {\sum }_{n = 1}^{N} \frac{{A}_{n}}{x {σ}_{n}} {e}^{\frac{- {( ln ( \frac{x}{{c}_{n}} ) )}^{2}}{2 {σ}_{n}^{2}}}$$

where N is the number of the decompositions i.e characteristic pulses, n is the index of the summation, A is amplitude, c is a center point and a is the standard deviation. As a result, each optically measured waveform or pulses may be represented by fifteen parameters when using 5 or other adjustable number of characteristic pulses of the decomposition, the values of parameters being estimated by a nonlinear curve fitting with constraints, for example.

An alternative to the Gaussian transform, per se, may be utilized. A reciprocal transform may also be used to make a pulse look like a Gaussian pulse.

An exponent transform may also be used to make a pulse look like a Gaussian pulse. Common exponents are square (square transform) and cube (cube transform) and corresponding roots.

Still another transform is a Box-Cox transform that may also be used to make a pulse look like a Gaussian pulse. A modification of the Box-Cox transform is Yeo-Johnson that may also be used to make a pulse look like a Gaussian pulse. These examples show that the measured glymphatic pulsation of the brain can be decomposed in a variety of manners for forming the G-index.

The pulse decomposition analysis (PDA) can be applied to a single pulse measured from the brain to extract several characteristic pulses that can be used to reconstruct the original one. Utilizing PDA in the fNIRS (functional Near InfraRed Spectral) signal analysis requires high enough sampling frequency to get good time resolution for the center position and pulse width parameters. A sampling frequency should be more than about 100 Hz, for example. A sampling frequency of about 500 Hz or higher may also be used, for example.

For each pulse, the PDA may be applied with the following algorithm to get five log-normal pulse with fifteen parameters of the Gaussian transform, which is here as an example
1. The algorithm guesses fifteen random numbers as starting points of the optimization algorithm (different starting point can result in different estimated parameters)
2. Based on the starting point, a minimization or optimization function with constraints, seeks for suitable parameters which follow the constraints
3. The estimated parameters are evaluated based on the error and correlation to the original pulse
4. If the evaluation criteria are not met, then the whole process is repeated.

The Gaussian transform, example of which are illustrated in FIGS. 6 and 7, may be used to provide five pulses a combination of which resembles the original pulse measured from the brain. From the five characteristic pulses, the first and third pulses represent systolic and diastolic pulse pressure, while the second and fourth ones are their reflections due bifurcation. With the fact that the reflected pulse from stiffened artery arrives earlier than that from elastic artery, the distance between the first and second pulses can be useful to estimate this brain stiffness. This assessment based on the optical measurement of the brain may be conducted in a segmented signal, e.g., the last about 2 minutes, for example, to get the results periodically.

As some individual pulses may be useless for the pulse decomposition, a pulse selection may be applied to remove unqualified pulses. Based on the pulse rate variability (like HRV in ECG), pulses with length outside the boundary of mean±3 times standard deviation may be discarded. Additional works such as correlating the pulse with a reference pulse can be used in order to collect a reference pulse, which may vary among the age group. As neurodegenerative diseases (NDDs) and other issues affect tissue stiffness, for example, a distance between the characteristic pulses can be used to quantify the degree of NDD severity. Instead of the NDD, some other reasons not necessarily pathological such as age, nutrition and/or lifestyle, for example, may affect the tissue stiffness.

The decompositions 1^{st} pulse, 2^{nd} pulse, 3^{rd} pulse, 4^{th} pulse and 5^{th} pulse have different heights and widths in FIGS. 6 and 7. The pulses 4^{th} pulse and 5^{th} pulse at the trailing edge are lower in a stiff brain than in an elastic brain, for example. Because there is a wide variety of possibilities how the waveform relates to the brain stiffness it is possible, in an embodiment, that the recognition of the brain stiffness from the waveform is based on artificial intelligence, neural network and/or machine learning methods, for example. Alternatively or additionally, the brain stiffness may be determined based on image processing of a curve of the waveform.

### Utilization of Statistical Moments

### Deviation

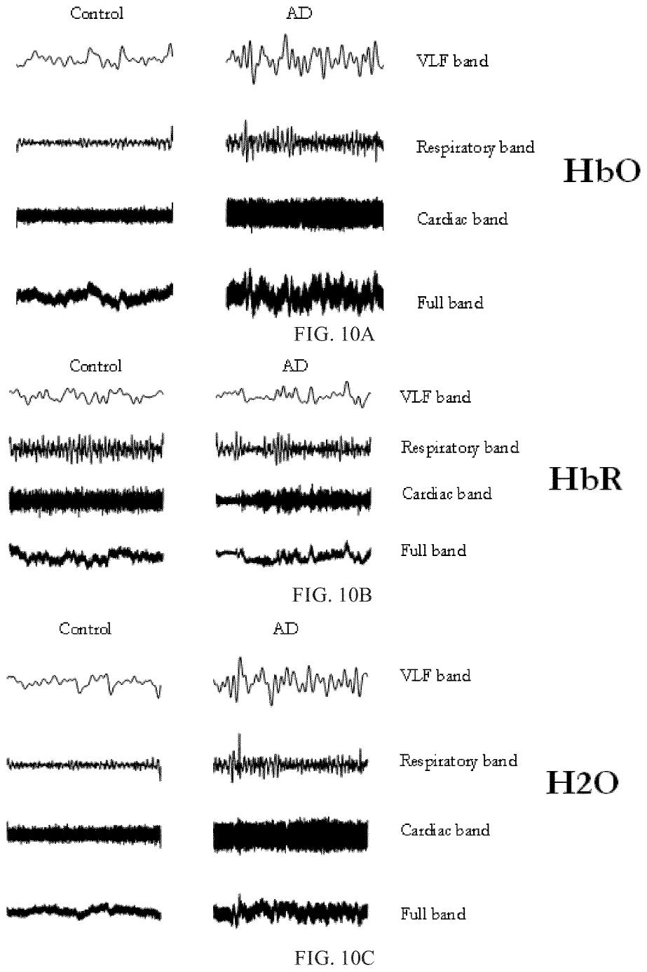

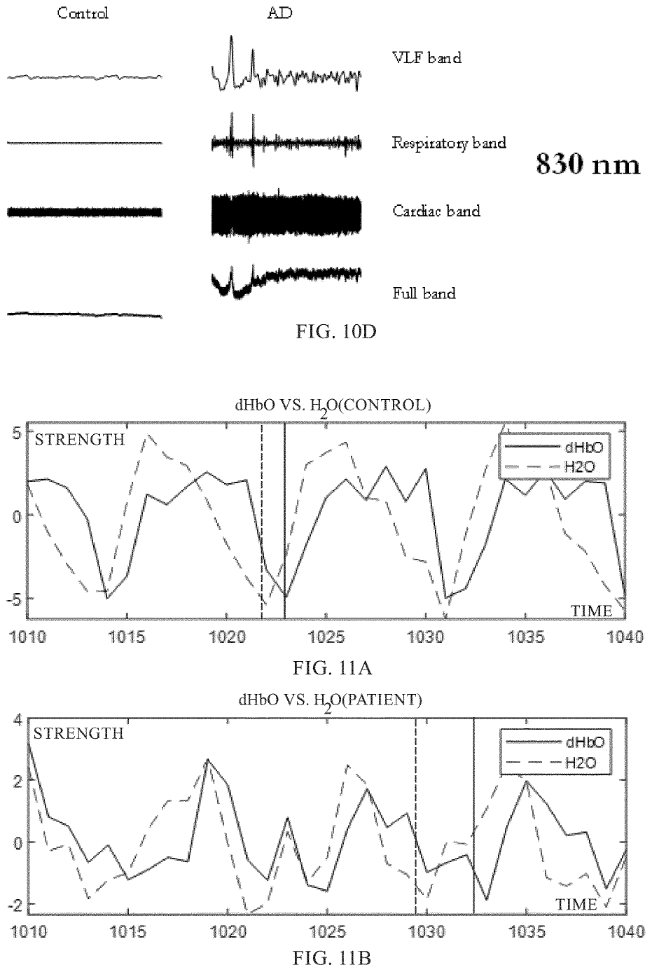

A variation around a mean (or center), i.e. standard deviation (SD), of blood oxygen level dependent (BOLD) may be used to separate patients with brain conditions such as Alzheimer's disease (AD) from a control group of people having healthy brain. FIGS. 8A to 8D illustrate examples of variation around the mean. The SD of the people with the brain condition (AD patients) is always larger than that of control group of healthy brain in a very low frequency band (VLF, 0.008-0.1 Hz), respiratory (0.1-0.6 Hz), and cardiac (0.6-5 Hz) bands. Similar procedures apply to oxy-haemoglobin HbO (see FIG. 10A), deoxy-hemoblobin HbR (see FIG. 10B), and water H₂O (see FIG. 10C) measurements. This is a method to separate patients of a brain condition such as AD from a reference based on a control group with the healthy brain using oxy-haemoglobin HbO and total-haemoglobin HbT in various bands. FIG. 10D presents an example that it is possible to use also only single wavelength (830 nm in this example) of a light source in order to realize a successful analysis. All measurements 10A to 10D show clearly that AD patients have larger variation around the mean than a control group with no AD do.

Variation around the mean may be performed as follows
1. Use MBLL to calculate concentration changes of HbO, HbR, and water, or electrical potential V signal in dcEEG
2. Apply various band-pass filter to select VLF (0.008-0.1 Hz), respiratory (0.1-0.6 Hz), and cardiac (0.6-5 Hz) bands
3. Calculate the standard deviation (SD) or variance from each concentration.
4. Normalize SD in coefficient of variation (CV)=SD/mean

### Water-Hemodynamic Coupling

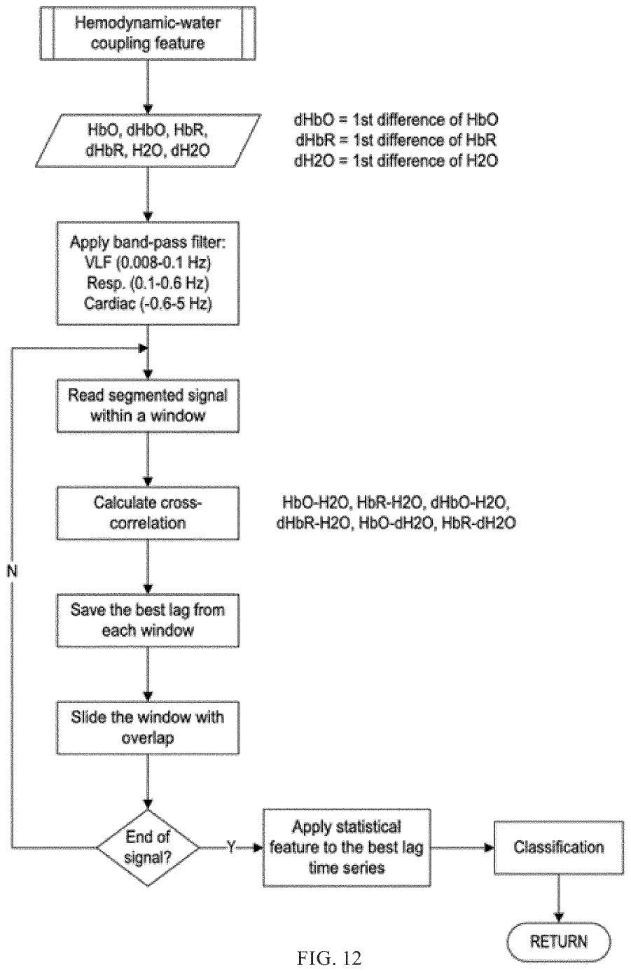

FIGS. 11A, 11B and 12 illustrate examples of analysis of the water-hemodynamic coupling. Based on the Monro-Kellie doctrine, the sum of volumes of brain, CSF, and intracranial blood is constant. It means there is a relationship between hemodynamic and water in the skull.

Hypothesis: if AD, other brain condition or a non-pathological state of the brain causes a problem in CSF and hemodynamic coupling, then a correlation analysis will reveal it.

The CSF or free water is calculated by subtracting about 80% of HbT from water concentration, because blood also contains water. Investigating on derivative −d(BOLD)/dt and CSF reveals that they have anti-correlation relationship with a certain lag. The analysis also involves the first difference of from both sides in VLF, respiratory, and cardiac bands.

Water-hemodynamic coupling may be determined in a following manner:
1. HbO, HbR, HbT, and water (H₂O) concentration and also their first difference, dHbO, dHbR, dHbT, and dH₂O may be used
2. The bands of interest may be VLF, respiratory, and cardiac bands
3. The following pairs may be used HbO-H₂O, HbR-H2O, HbT-H₂O, dHbO-H₂O, dHbR-H₂O, dHbT-H₂O, HbO-H₂O, HbR-dH₂O, and HbT-dH₂O, and also with dcEEG signals in all bands (VLF, resp, card, delta(0.2-4 Hz), theta(4-7 Hz), alpha (8-12 Hz), beta(20-40 Hz), gamma(40-70) and their envelopes)
4. Cross correlation may be applied to each pair for segmented signal using pre-defined window, and a minimum width is a longest period in each band, while a maximum is approximately 1.5 times of the minimum.
   a. VLF=VLF: 120, 140, 160, 180 seconds, for example
   b. Respiratory: 10, 12, 14, 16 seconds, for example
   c. Cardiac: 2, 2.4, 2.6, 3 seconds, for example
5. The window may be shifted such that it has overlap of about 25%, 50%, and 75%
6. The lag values at the best correlation coefficient from each window may form a time-series data for further analysis
7. This time-series may be subjected to statistical analysis, such as mean, SD, median, IQR (interquartile range), skewness, and kurtosis.

In addition, an SD and IQR of lag variation profile involving HbR agrees with mini mental state exam (MMSE) score. It also indicates that a control group of healthy brain have more variation in a lag profile than the AD patients and others with a brain condition do.

Fractional Amplitudes of Physiological Fluctuation (fAPF)

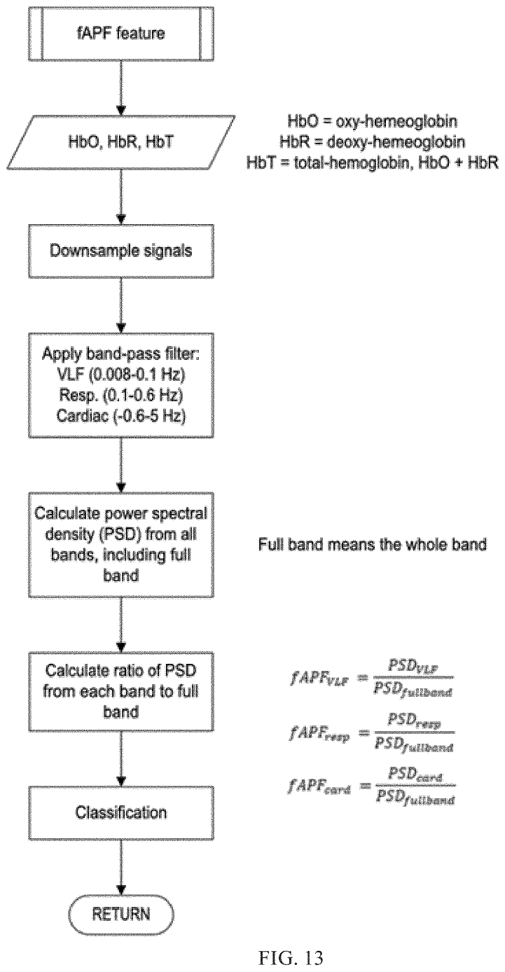

An example of a flow chart of the fAPF 1 is illustrated in FIG. 13. In this method, a ratio of the PSD (Power Spectral Density) between a low frequency band and the full band is computed. The method of computing a ratio between a part of a band and the whole band is known, per se, for example in the functional magnetic resonance imaging community but a person skilled in the art is not familiar with it use or applicability in the optical measurements of the brain. Instead of using the suggested low frequency band, the fAPF (fractional amplitude of physiological fluctuation) uses very low frequency (VLF, about 0.008 Hz to about 0.1 Hz), respiratory (about 0.1 Hz to about 0.6 Hz), and cardiac (about 0.6 Hz to about 5 Hz) bands. The VLF band reflects vasomotor response, which relates to constriction and dilation of blood vessel.

Mathematically this may be expressed as follows:

$${fAPF}_{VLF} = \frac{{PSD}_{VLF}}{{PSD}_{fullband}}$$

$${fAPF}_{resp} = \frac{{PSD}_{resp}}{{PSD}_{fullband}}$$

$${fAPF}_{card} = \frac{{PSD}_{card}}{{PSD}_{fullband}}$$

where PDS_{VLF} is the very low frequency band, PDSᵣₑₛₚ is the respiratory band, PDS_{card} is the cardiac band and PDS_{ulband} is the fulband about 0.008 Hz to about 5 Hz.

An algorithm of the fAPF measurement may be as follows
1. Calculate power spectral density (PSD) of HbO, HbR, and HbT from VLF band, respiratory band, cardiac band, and the full band used in the measurement.
2. Calculate area under the curve (AUC) of the power spectral density using trapezoid integration, for example
3. Divide the power spectral density of VLF, respiratory, and cardiac bands by the power spectral density of the full band to get the fAPF from corresponding band.

Experiments on the fNIRS signals measured from both AD patients and/or other patients with different brain conditions and a control group with the healthy brain are promising. The fAPF of HbT in cardiac band can be used to separate patients with a brain condition from a control group with the healthy brain. Here like also in other parts of this document, the brain condition may mean a brain disease like AD, a mental health issue, lifestyle effect in the brain and/or injury, for example.

### PSD Ratio of High to Low Band

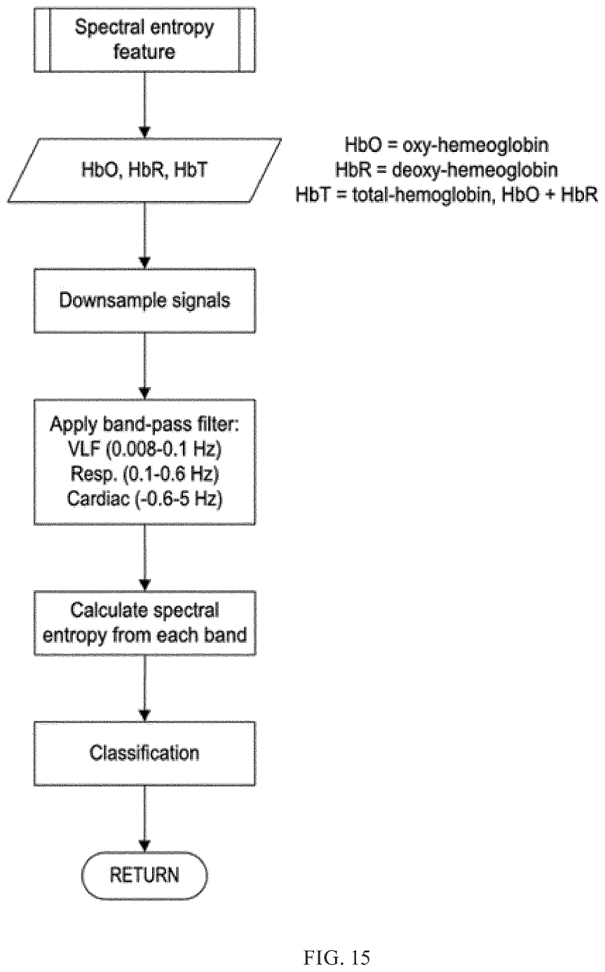

An example of a flow chart of the PSD ratio of high and low band is illustrated in FIG. 15. Another use of the power spectral density relates to characterization of broadband random signals. In the fNIRS domain, the signal amplitudes among subjects vary a lot and comparing the (Power Spectral Density) values among subjects may be sometimes inaccurate. Therefore in an embodiment, a ratio between the PSDs of high and low frequency bands is a suitable way to normalize this value. This method was inspired by HRV analysis in frequency domain.

Mathematically this may be expressed as follows:

$${Ratio}_{VLF} = \frac{{PSD}_{VLF \_ high}}{{PSD}_{VLF \_ low}}$$

$${Ratio}_{resp} = \frac{{PSD}_{resp \_ high}}{{PSD}_{resp \_ low}}$$

$${Ratio}_{card} = \frac{{PSD}_{card \_ high}}{{PSD}_{card \_ low}}$$

There is an example of the high and low PSDs for VLF below. The high and low of the respiratory band and cardiac band are defined in a corresponding manner.

The PSD ratio of high to low frequency band may be as follows
1. Calculate threshold to separate low and high band within the VLF band, respiratory band, and cardiac bands
   a. Log-scale may be used instead of a linear scale
   b. For each band, there are two values as the frequency limits
   c. Centre point may be calculated as the mean of log(low_limit) and log(high_limit).
   d. Convert this log-scale to linear scale using 10 raised to power of centre value.

### Example of a VLF Band

i. Low and high limits are 0.008 Hz and 0.1 Hz, respectively
ii. Centre point=(log(0.008)+log(0.1))/2=−1.5485
iii. In linear scale it is about 0.0283 Hz.
iii. Calculate PSD from each frequency band
3. Using the center point, each band may be divided into low and high frequency band
4. AUC of low and high frequency bands may be computed using trapezoid integration, for example, to get PSD_low_band and PSD_high_band respectively.
5. Divide the PSD of the high frequency band by the PSD of the low frequency band to get the feature.

### Sample Entropy

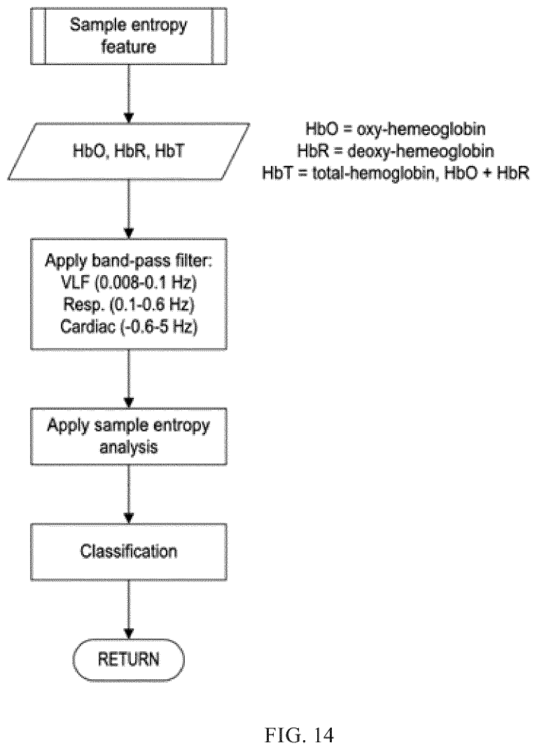

An example of a flow chart of the sample entropy is illustrated in FIG. 14. Entropy measures randomness of a time series data without prior knowledge about the source of the data generator. Consequently, it can be used to estimate regularity of the data. Unfortunately, entropy estimation requires the whole time series, which is not normally available. However, an estimate of this kind of partial signal may be formed using an algorithm called approximate entropy, a person skilled in the art being familiar with the algorithm, per se. A sample entropy may additionally be used to improve approximate entropy algorithm.

Sample entropy may be as follows
1. Sample entropy may be applied together with a Chebychev distance measure to HbO, HbR, and HbT in different bands
2. Compare sample entropy values of different age groups may be compared.

Experiments on HbO, HbR, and HbT using sample entropy with Chebychev distance measure are promising. The sample entropy scores are gradually increasing and proportionally to the age of the group, except for HbT in age less than 30 years old. It might indicate that the older the person, the more random the fluctuation of Hb, HbR, and HbT is.

### Spectral Entropy

An example of a flow chart of the spectral entropy is illustrated in FIG. 15. Spectral entropy is a measure of the spectral power distribution of a signal based on the Shannon entropy. Thus, it applies the Shannon entropy to the signal's normalised power distribution. The spectral entropy analysis on MREG, EEG, and fNIRS signal revealed electrophysiological and hemodynamic changes in drug-resistant epilepsy.

The spectral entropy calculation starts from normalised power spectral density (PSD). There are many methods to get the PSD, e.g., FFT (Fast Fourier Transform) and a periodogram. The spectral entropy is simply applying Shannon entropy to probability distribution of the normalised PSD. The spectral entropy can be applied to a raw fNIRS signal and concentration changes.

Spectral entropy may be as follows
1. Use signal sampled at 10 Hz instead of 800 Hz
2. A band pass filter (BPF) may be applied to select the interested band: VLF, respiratory, or cardiac
3. Spectral entropy may be calculated using pentropy function in Matlab or the like
4. Compare spectral entropy values may be compared between patients with brain issues and a control group with a healthy brain using t-test at 0.05 significant level, for example.

Here again, the brain issue may mean a brain disease like AD, a mental health issue, lifestyle effect in the brain and/or injury, for example.

### Cardiorespiratory Envelope Modulation (CREM)

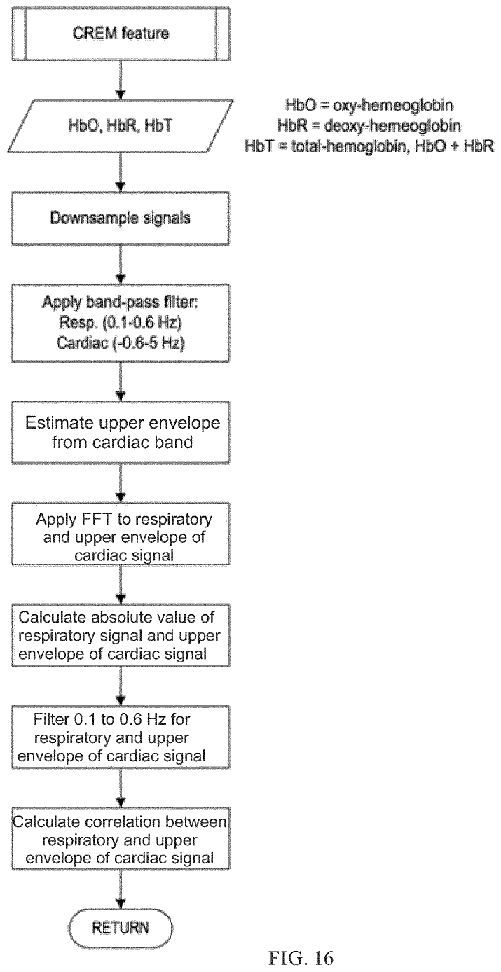

An example of a flow chart of the CREM is illustrated in FIG. 16. Analysis on blood oxygen level dependent (BOLD) signal shows that respiratory band modulate signals in cardiac band. The same method may be applied to HbO, HbR, and HbT.

An algorithm the CREM may be as follows:
1. Apply BPF (Band Pass Filtering) to select respiratory and cardiac band for the analysis
2. Estimate upper envelope of the cardiac signal
3. Apply FFT (Fast Fourier Transform) to the respiratory signal and the upper envelope of the cardiac signal
4. Calculate absolute value of the FFT from step 3 for respiratory signal and the upper envelope of the cardiac signal
5. Limit the results in respiratory band and/or the upper envelope of the cardiac signal to about 0.1 Hz to about 0.6 Hz
6. Calculate correlation between absolute values of the respiratory signal and the upper envelope of the cardiac signal limited to about 0.1 Hz to about 0.6 Hz.

### Phase Transfer Entropy

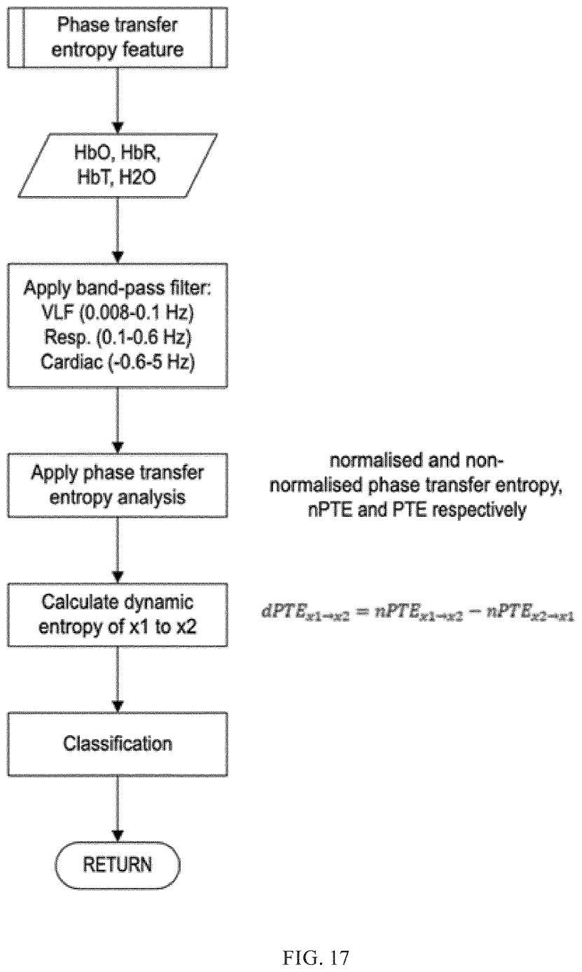

An example of a flow chart of the phase transfer entropy is illustrated in FIG. 17. It uses a mean of phase transfer entropy to get a differential phase transfer entropy dPTE (normalised) and PTE values. Evaluate coupling for the following pairs is evaluate: HbO-HbR, HbO-H₂O, HbR-H₂O, and HbT-H₂O.
2. Using dPTE (normalised values), a transfer from both directions gives the same p-value
3. Using PTE, a transfer from both directions may have different p-value, it even changes from significant to non-significant and vice versa
4. Significant results from 10 Hz:
a. HbO-HbR at cardiac band (L) à 0.0277
b. HbT-H₂O at respiratory band (L) à 0.0215
5. Significant results from 800 Hz:
a. HbO-HbR at VLF band (L) à 0.0279
b. HbO-H₂O at VLF band (R) à 0.0420
c. HbO-H₂O at respiratory band (L) à 0.0486.

Mathematically the phase transfer entropy can be expressed as:

dPTE_{x1→x2}=nPTE_{x1→x2}−nPTE_{x2→x1}.

Here the non-integer numbers after the symbol “à” refer to a p-value which indicates a probability of error. When the p-value is below 0.05, the result is significant.

An acceleration signal recorded by accelerometer is sensitive to vibration; thus, the raw signal may look noisy. For this reason, a low-pass filter may be used to remove unnecessary spike due to this property. Then, the signal may be utilized.

Body movement can be estimated by placing a 3D accelerometer on the body, especially on the waist. It may be placed on the wrist and even finger. Related to sleep study, where the body position is limited, the easiest way to estimate body position is placing the sensor on the chest. However, it is somehow not practical for the G-index measurement because the optical measurement arrangement 120 and the potential EEG measurement is worn on the head and perhaps on the forehead. For this reason, it is more practical to put the accelerometer on the forehead.

Alternative body positions during sleep are supine with head at the centre, supine with the head to the left or right, and laying on the left or right side. It means, there are five possible positions of the head during sleeping, but they correspond to three body positions.

Using a 3D accelerometer, three different axes of the movement can be assessed. Related to the head movement, the axis parallel to the left and right movement can be the dominant signal, while the other serve as secondary ones. Another secondary signal is single magnitude area of the 3D acceleration signal. When a person is laying in the side, the forehead is about perpendicular to the vertical axis. The situation is slightly different from supine position with the head to left or right. The angle difference should be enough to separate laying on the side from supine. To make the prediction more robust, a machine learning algorithm may be used. Features may be extracted based on the body position and its transition.

Since the body position is tracked during sleeping, the signal is processed based on a pre-defined window with certain overlap, e.g., about 25%. Since the body movement and its transition time varies among different people, the window width may be determined carefully. The width should not be too narrow such that the acceleration signal is too short to capture the movement or too wide such that there are several important movements in the same window. As a result, the data processing unit 150 may determine the G-index also based on the position or positions of the subject. The reference for the measurement can be acquired by measuring a control group in one or more defined positions.

A set of raw signals from a glymphometer may comprise four fNIRS signals from different wavelengths, EEG, acceleration signals, and other possible modalities, for example. Due to hardware complexity and other reasons, the signals may contain unwanted spikes that should be removed, otherwise the concentration calculation will be affected. These spikes appear at random places and they may be either up- or down-spikes.

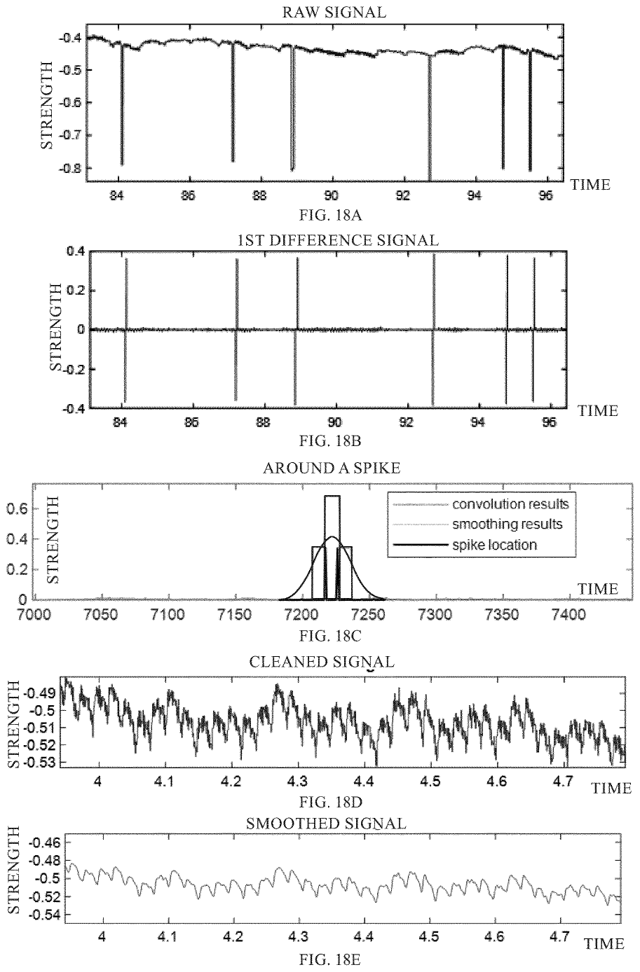

FIGS. 18A to 20 illustrate examples of how to pre-process signals before pulse analysis. FIG. 18A illustrates an example of a raw signal for the G-index measurement. FIG. 18B illustrates an example of pre-processed fNIRS signal, where an unwanted spikes of the raw signals have been replaced by vertical lines using a derivative or difference of the spikes. The slope of the spikes is either positive or negative.

Sometimes there is another unwanted spike within the existing unwanted spike. The phenomenon results in more than two vertical lines. This may lead to a problem to identify a single point to mark the location of the unwanted spike. For this reason, these vertical lines may be combined into a single pulse and convolution with a rectangular pulse, N=20, for example, followed by applying MA filter (N=25), for example. This creates a nice gaussian-like shape pulse at the unwanted spikes' locations. The gaussian-like pulse then may denote a location of the unwanted spike.

Next, a gap between the vertical lines have been replaced by a linear line between the extreme points of the gap for making the signal curve continuous. That is, each unwanted spike is replaced with a linear line based on a linear line equation between two points. Finally, the signal may be smoothed by a moving average filter.

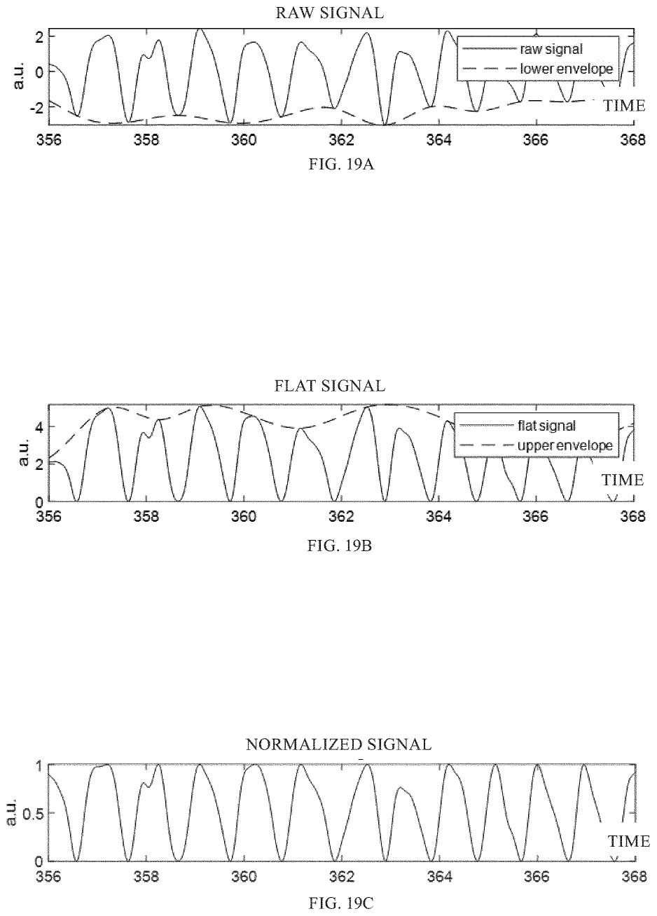

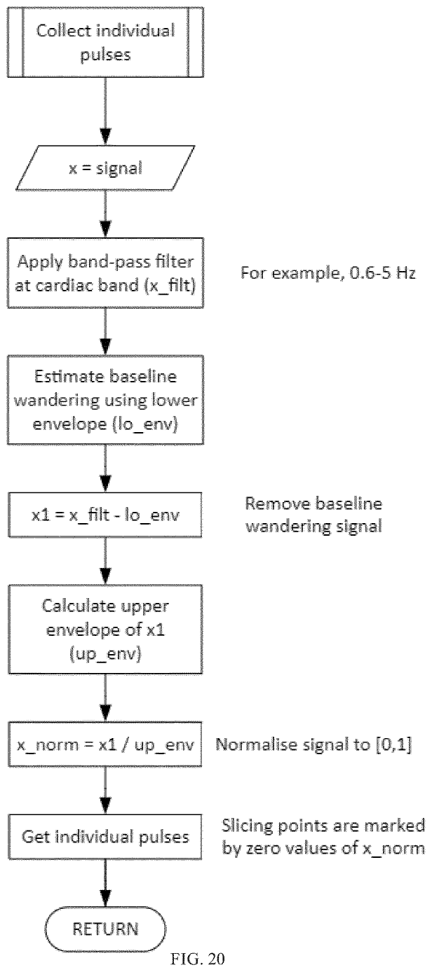

FIGS. 19A to 19C illustrate examples of a baseline straightening signal normalizing. Band-pass filter, e.g., about 0.5 Hz to about 3 Hz, may be applied to clean_the raw_signal, that is, to remove unnecessary frequency outside the cardiac band. Then baseline wandering is estimated using an upper envelope and a lower envelope of the raw signal. To eliminate or reduce the baseline wandering, estimated_baseline_wandering based on the lower envelope is subtracted from the raw signal. The upper envelope may be estimated from signal_without_baseline_wandering. Dividing signal_without_baseline_wandering may be divided with the upper envelope to normalise it to a range [0,1], for example. Each pulse of the normalized signal may be separated by slicing the signal at points where the signal is zero. The steps of FIGS. 19A to 19C are also illustrated in FIG. 20 as a flow chart.

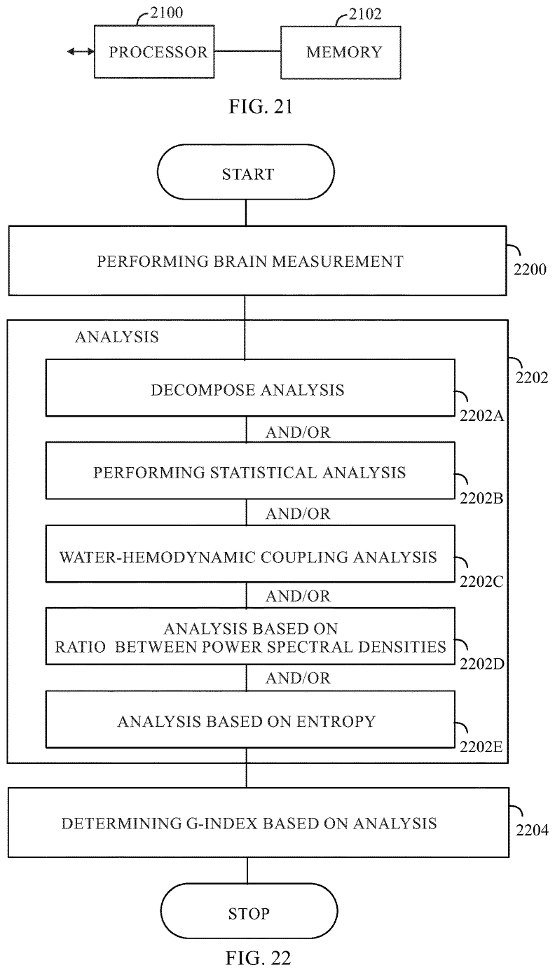

FIG. 21 illustrates an example of the data processing unit 150 which is shown in FIG. 1. The data processing unit 150 comprises one or more processors 2100 and one or more memories 2102 including computer program code.

The term “computer” includes a computational device that performs logical and arithmetic operations. The data processing unit 150 is a computer. For example, a “computer” may comprise an electronic computational device, such as an integrated circuit, a microprocessor, a mobile computing device, a laptop computer, a tablet computer, a personal computer, or a mainframe computer. A “computer” may comprise a central processing unit, an ALU (arithmetic logic unit), a memory unit, and a control unit that controls actions of other components of the computer so that steps of a computer program are executed in a desired sequence. A “computer” may also include at least one peripheral unit that may include an auxiliary memory (such as a disk drive or flash memory), and/or may include data processing circuitry.

A user interface 152, which is shown in FIG. 1, means an input/output device and/or unit. Non-limiting examples of a user interface include a touch screen, other electronic display screen, keyboard, mouse, microphone, handheld electronic game controller, digital stylus, display screen, speaker, and/or projector for projecting a visual display.

The one or more memories 2102 and the computer program code configured to, with the one or more processors 2100, cause apparatus at least to perform the method steps of the analysis a flow chart of which is shown in FIG. 22.

FIG. 22 is a flow chart of the analysis method. In step 2200, an optical measurement of intracranial dynamics including the intracranial neuronal and fluids dynamics, brain tissue pulsation and/or glymphatic activity through the cranium by an optic measurement arrangement 120.

In step 2202, at least one of the following analyses is applied, by the data processing unit 150, to electrical signals, the electrical signals being received from the optic measurement arrangement 120 and carrying information on said intracranial dynamics through the cranium in forms of pulses:
decomposing 2202A at least one of the pulses of the of said intracranial dynamics into a plurality of characteristic pulses for processing with at least one relation between the characteristic pulses,
at least one moment of statistical analysis of the pulses of said intracranial dynamics is determined in step 2202B,
a water-hemodynamic coupling is determined in step 2202C from the pulses of said intracranial dynamics based on a definition that a sum of volumes of the brain, celebrospinal fluid and intracranial blood is constant,
a ratio between a power spectral density of a fraction of a whole measured frequency band and a power spectral density of the whole measured frequency band is determined in step 2202D,
entropy relating to the electrical signals is estimated in step 2202E.
   In step 2204, a G-index is determined based on said analysis of the electrical signals, which describes a state of brain function, and a reference, which is based on a corresponding analysis of at least one control group with known and/or estimated intracranial dynamics, the G-index representing a relative intracranial dynamics of the brain with respect to the reference.

The method shown in FIG. 22 may be implemented as a logic circuit solution or computer program. The computer program may be placed on a computer program distribution means for the distribution thereof. The computer program distribution means is readable by a data processing device, and it encodes the computer program commands, carries out the measurement and analysis and optionally controls the processes on the basis of the measurement.

The computer program may be distributed using a distribution medium which may be any medium readable by the controller. The medium may be a program storage medium, a memory, a software distribution package, or a compressed software package. In some cases, the distribution may be performed using at least one of the following: a near field communication signal, a short distance signal, and a telecommunications signal.

It will be obvious to a person skilled in the art that, as technology advances, the inventive concept can be implemented in various ways. The invention and its embodiments are not limited to the example embodiments described above but may vary within the scope of the claims.

What is claimed is:

1. An analyzing method for intracranial dynamics, the method comprising
performing an optoelectronic measurement of the intracranial dynamics including intracranial neuronal and fluids dynamics, brain tissue pulsation and/or glymphatic activity through the cranium by an optoelectronic measurement arrangement;
applying, by the data processing unit, at least one of the following analyses to electrical signals, the electrical signals being received from the optoelectronic measurement arrangement and carrying information on the intracranial dynamics in forms of pulses:
decomposing at least one of the pulses of the intracranial dynamics into a plurality of characteristic pulses for processing with at least one relation between the characteristic pulses,
determining) at least one moment of statistical analysis of the pulses of the intracranial dynamics,
determining a brain fluid, particularly water-hemodynamic, coupling from the pulses of the intracranial dynamics based on a definition that a sum of volumes of the brain tissue, cerebrospinal fluid and intracranial blood is constant,
determining a ratio between a power spectral density of a fraction of a whole measured frequency band and a power spectral density of the whole measured frequency band,
estimating entropy relating to the electrical signals; and
determining a G-index based on said analysis of the electrical signals and a reference, which is based on an analysis of at least one control group with known and/or estimated intracranial dynamics, the G-index representing a relative dynamics of the brain.

2. The method of claim 1, the method further comprising
directing, by the optic measurement arrangement, optic radiation toward the brain through the cranium;
receiving, by the optic measurement arrangement, the optic radiation interacted with the brain;
converting, by the optic measurement arrangement, the optic signals into electric signals;
detecting, by a data processing unit, pulses of the intracranial dynamics.

3. The method of claim 1, the method further comprising performing the decomposition of the at least one of the pulses of the of the intracranial dynamics into the plurality of the characteristic pulses by converting the at least one pulse into a predetermined number of characteristic pulses by a Gaussian decomposition, the predetermined number being adaptable; and forming the G-index based on at least one temporal distance between the characteristic pulses.

4. The method of claim 1, the method further comprising performing the determination of the at least one moment of the statistical analysis of the pulses of the intracranial dynamics as a variation of a moment with respect to an average of the moment.

5. The method of claim 1, the method further comprising performing the determination of the water-hemodynamic coupling from the pulses of the intracranial dynamics based on the definition that the sum of volumes of the brain, celebrospinal fluid and intracranial blood is constant by determining oxy-haemoglobin, deoxy-haemoglobin and total haemoglobin and their difference.

6. The method of claim 1, the method further comprising performing the determination of the ratio between the power spectral density of the fraction of the whole measured frequency band and the power spectral density of the whole measured frequency band by
determining a fractional amplitude of physiological fluctuation based on a ratio of at least one of very low frequency band to a full band 0.008 Hz to 0.1 Hz, respiratory band and cardiac band to a full band covering at least 0.008 Hz to upper end of the cardiac band used in the measurement.

7. The method of claim 1, the method further comprising performing the estimation of the entropy in a time domain and/or in a frequency domain of the electrical signals.

8. The method of claim 1, the method further comprising measuring electroencephalographic signal of the brain; and applying, by the data processing unit, at least one of the following analyses to the electroencephalographic signal:
estimating entropy relating to the electroencephalographic signal,
determining a ratio between a power spectral density of a fraction of a whole measured frequency band and a power spectral density of the whole measured frequency band of the electroencephalographic signal; and
combining information from the electroencephalographic and corresponding analysis of the optical measurements for forming the G-index.

9. An analyzing apparatus of intracranial dynamics; wherein
apparatus comprises one or more processors; and
one or more memories including computer program code;
the one or more memories and the computer program code configured to, with the one or more processors, cause apparatus at least to perform the method steps of claim 1.
